(19) United States
(12) Patent Application Publication
(10) Pub. No.: US 2021/0156830 A1
(43) Pub. Date: May 27, 2021

(54) CHARACTERIZATION OF SOLID CATALYSTS

(71) Applicant: Saudi Arabian Oil Company, Dhahran (SA)

(72) Inventors: Manal Al-Eid, Dhahran (SA); Lianhui Ding, Dhahran (SA)

(73) Assignee: Saudi Arabian Oil Company, Dhahran (SA)

(21) Appl. No.: 16/696,828

(22) Filed: Nov. 26, 2019

(51) Int. Cl. G01N 30/66; G01N 25/18; G01N 1/28

(52) U.S. Cl. CPC G01N 30/66; G01N 2030/008; G01N 1/28; G01N 25/18

(57) ABSTRACT

Examples described herein provide a method for characterizing a catalyst in a chemisorption unit. The method includes treating a catalyst sample with gas blend comprising ammonia in an inert gas and performing a first temperature programmed desorption (TPD) to desorb the ammonia from the catalyst sample. A temperature programmed reduction (TPR) of the catalyst sample is performed with hydrogen. The catalyst sample is treated after the TPR with a gas blend comprising ammonia in an inert gas. A second temperature programmed desorption is performed to desorb the ammonia from the catalyst sample.

### BACKGROUND

Catalysts are used in the production of fuels, petrochemicals, polymers, and many other chemical products. Many of these catalysts are heterogeneous, for example, having active sites, such as a metal, on a support, such as a zeolite or an aluminum oxide. Heterogeneous catalysts often function by facilitating reactions between species that have reacted to form chemical bonds with the surface, termed chemically adsorbed species. As used herein, chemical adsorption is also termed chemisorption. The effective design and utilization of the catalysts may be aided by characterizing the number of active sites and the acidity of the support.

The characterization of catalysts may also be performed by chemisorption. In this application, molecules that are used as molecular probes are chemisorbed and desorbed from the surface. Prior to adsorption, the molecular probes are termed adsorptive molecules. Once adsorbed, or chemically reacted with the surface, the molecular probes are termed adsorbate molecules. As the molecular probes react with the surface through chemical reactions, they are specific to active sites. For example, basic molecules, such as ammonia, react with acidic sites on a heterogeneous catalyst. Accordingly, the amount of the molecular probes that are adsorbed, the temperature at which they are adsorbed, and the temperature at which they are desorbed may be analyzed to determine the characteristics of the surface.

### SUMMARY

An embodiment described herein provides a method for characterizing a catalyst in a chemisorption unit. The method includes treating a catalyst sample with gas blend including ammonia in an inert gas and performing a first temperature programmed desorption (TPD) to desorb the ammonia from the catalyst sample. A temperature programmed reduction (TPR) of the catalyst sample is performed with hydrogen. The catalyst sample is treated after the TPR with a gas blend including ammonia in an inert gas. A second temperature programmed desorption is performed to desorb the ammonia from the catalyst sample.

In an aspect, the method includes loading the catalyst sample in the chemisorption unit, and drying the catalyst sample by flowing the inert gas over the catalyst sample while ramping a temperature of the catalyst sample from about 50° C. to about 500° C. at about 10° C./min.

In an aspect, treating the catalyst sample with a gas blend includes flowing a blend of 10% ammonia in helium at a flow rate of about 30 cc/min for about 35 minutes at about 50° C.

In an aspect, performing the first temperature programmed desorption includes flowing a carrier gas over the catalyst sample, wherein the carrier gas includes helium, and ramping a temperature of the catalyst sample from about 50° C. to about 550° C. at about 10° C./min. Data is collected from a thermal conductivity detector to measure ammonia desorbed at a temperature during the ramping.

In an aspect, an amount of the ammonia desorbed from the catalyst sample during the first TPD versus time is measured by plotting a response from a thermal conductivity detector versus temperature. In an aspect, a total acidity of the catalyst sample is measured by integrating an area of a measured response from the thermal conductivity detector. In an aspect, a relative acid strength of the catalyst is determined by comparing peak locations of the ammonia released from other catalyst samples. In an aspect, the catalyst sample is cooled under a flow of the inert gas.

In an aspect, an amount of hydrogen adsorbed by the catalyst sample is measured during the TPR by measuring an amount of hydrogen removed from a carrier gas. In an aspect, performing the TPR of the catalyst sample with hydrogen includes flowing a carrier gas over the catalyst sample, wherein the carrier gas includes a gas blend of 5% hydrogen in argon, ramping the temperature of the catalyst sample from about 50° C. to about 800° C. at about 10° C./min, and collecting data from a thermal conductivity detector to measure an amount of hydrogen removed from the carrier gas at a temperature during the ramping.

In an aspect, an activity of the catalyst sample is determined by integrating peaks of the hydrogen adsorbed during the TPR. In an aspect, a number of active sites of the catalyst sample are determined by determining a number of peaks in a plot of the TPR. In an aspect, at least a portion of metal sites are dispersed by performing the TPR.

In an aspect, treating the catalyst sample after the TPR includes flowing a gas blend including 10% ammonia in helium over the catalyst sample at a flow rate of about 30 cc/min for about 35 minutes at about 50° C. In an aspect, performing the second temperature programmed desorption includes flowing a carrier gas over the catalyst sample, wherein the carrier gas includes helium, ramping the temperature from about 50° C. to about 550° C. at about 10° C./min, and collecting data from a thermal conductivity detector to measure ammonia desorbed at a temperature during the ramping.

In an aspect, an amount of the ammonia desorbed from the catalyst sample during the second TPD is measured versus time by plotting a response from a thermal conductivity detector versus temperature. In an aspect, a total acidity of the catalyst sample is measured by integrating an area of a measured response from the thermal conductivity detector. In an aspect, a relative acid strength of the catalyst is determined by comparing peak locations of the ammonia released from other catalyst samples. In an aspect, a change in acid strength of the catalyst sample is determined by comparing results from the second temperature programmed desorption to results of the first temperature programmed desorption.

### BRIEF DESCRIPTION OF DRAWINGS

FIG. 1 is a process flow diagram of a method for characterizing a catalyst by performing an acidity analysis both before and after a reduction analysis, in accordance with an embodiment.

FIG. 2 is a block diagram of a chemisorption unit that may be used in embodiments.

FIG. 3 is a screenshot of the parameters used for the drying step (T1), in accordance with an embodiment.

FIG. 4 is a screenshot of the parameters used for treating the catalyst sample with ammonia (T2), in accordance with an embodiment.

FIG. 5 is a screenshot of the parameters used for performing the first temperature programmed desorption of ammonia (T3), in accordance with an embodiment.

FIG. 6 is a screenshot of the parameters used for the hydrogen temperature program reduction of the catalyst sample (T4), in accordance with an embodiment.

FIG. 7 is a screenshot of the parameters used for the second ammonia treatment step of the catalyst sample (T5), in accordance with an embodiment.

FIG. 8 is a screenshot of the parameters used for performing the second temperature programmed desorption of ammonia (T3), in accordance with an embodiment.

FIG. 9A is a plot of the first and second temperature programmed desorption curves for ammonia for the Pt-KL catalyst sample.

FIG. 9B is a plot of the temperature programmed reduction of the Pt-KL catalyst.

FIG. 10A is a plot of the first and second temperature program desorption of ammonia from a Ga-ZSM 5 catalyst.

FIG. 10B is a plot of the temperature programmed reduction of the Ga-ZSM 5 catalyst.

### DETAILED DESCRIPTION

Embodiments described herein provide a test method to characterize the acidity of a catalyst sample, before metals in the catalysts are reduced, followed by determining the metal distribution and number of active sites after measuring the acidity. Further, the method determines the acidity change after the metals in the catalyst are reduced and dispersed. The method is implemented by first characterizing a solid sample by ammonia temperature programmed desorption (NH3-TPD), then characterizing the same sample by hydrogen temperature programmed reduction (H2-TPR). After the H2-TPR measurement is completed, the same sample is characterized by a second NH3-TPD measurement.

Heterogeneous catalysts are generally formed from solid supports, such as zeolites or alumina, with metal compounds supported on the solid supports. To prepare the catalyst for use, it is often reduced to form metal grains on the solid support. Many catalysts are supported on zeolite structures, due to the open pore structure of the zeolites. Generally, a zeolite is an aluminosilicate compound commonly mainly made from Si, Al, 0, and metals including Ti, Sn, Zn, and the like. Different zeolites are classified by their structure, and are usually synthetically formed for catalytic purposes to avoid contamination from other natural materials

One such catalyst is Pt/KL, which has an active form of Pt⁰ sites supported on an L type zeolite. Zeolite L, also termed LTL, has an open pore structure having a hexagonal crystal system. The zeolite L material may be treated with a platinum precursor, for example, by impregnation or ion-exchange, such as platinum chloride, among others, to form the Pt/KL catalyst. After treatment, the platinum compound may be calcined to remove other ions, such as the chlorine, forming platinum oxide in the lattice structure. The platinum oxide may then be reduced, such as by hydrogen in chemisorption, to form the platinum sites in the active catalyst. Pt/KL catalyst may be used for aromatization of light alkanes (such as propane to hexane), as well as the production of aromatic hydrocarbons from naphtha.

Another zeolite supported catalyst is Ga-ZSM-5. ZSM-5 is an aluminosilicate zeolite having a high silicon to aluminum ratio. The ZSM 5 has a pentasil based crystal system providing smaller pores than the Pt/KL catalyst. The high silicon to aluminum ratio increases the acid strength of the zeolite, increases its activity for certain reactions, such as hydrocarbon isomerization and alkylation. The Ga-ZSM-5 catalyst has a number of applications, such as the direct conversion of methane to liquid hydrocarbons, and the catalytic aromatization of light alkanes, naphtha, and dilute ethylene streams.

Both of these catalysts have been characterized using the techniques described herein, as discussed further with respect to the examples. Many other types of catalysts could benefit from the techniques including, for example, other heterogeneous supported catalysts.

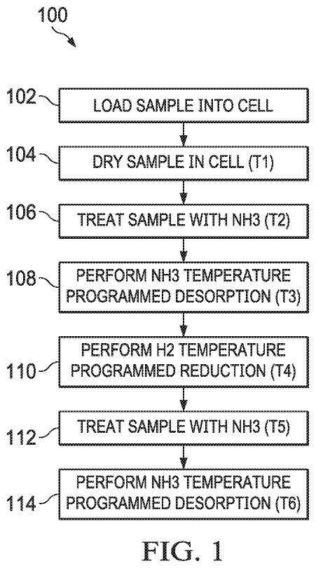

FIG. 1 is a process flow diagram of a method 100 for characterizing a catalyst by performing an acidity analysis both before and after a reduction analysis, in accordance with an embodiment. The entry of the conditions, and the setup of the instrumentation is discussed further with respect to the examples below.

The method 100 begins at block 102, when a catalyst sample is loaded into a cell. The cell is placed into a chemisorption analysis system. Any number of chemisorption analysis systems may be used, including the ChemStar from Quantachrome instruments, or the AutoChem 2920 or 2950 instruments from Micromeritics Instrument Corporation 2950, among others.

At block 104, the catalyst sample in the cell is dried, for example, by flowing an inert gas, such as helium, nitrogen, or argon, over the catalyst sample at an elevated temperature. In an embodiment, the catalyst sample is dried at about 500° C. for about 35 minutes using helium as the inert gas. The temperature is then dropped to about 50° C. The main purpose of drying is to remove the moisture and volatile materials on the sample to avoid interference with the analysis (for example, TCD signals), while maintaining the physiochemical properties of the sample. The highest drying temperature depends on the sample stability under the thermal treatment, which will be determined by other analysis and studies. For typically zeolites, drying below 550° C. is recommended.

At block 106, the catalyst sample in the cell is treated with ammonia. The ammonia reacts with acidic sites on the catalyst support. In an embodiment, the helium of the carrier gas is replaced with a gas blend of about 10 vol. % ammonia in helium. Depending on the catalyst sample involved, the amount of ammonia used may be about 5 vol. %, about 10 vol. %, or about 20 vol. %. The treatment may take place for about five minutes, about 30 minutes, about one hour, about five hours, or longer.

At block 108, a temperature programmed desorption of the ammonia is performed. This may be performed under a flow of helium, as the temperature is increased from about 50° C. to about 700° C. The ammonia released is detected by a thermal conductivity detector in the effluent stream from the sample chamber.

This size of the peaks indicate how much ammonia is desorbed. The peak area can be integrated, for example, automatically by the analysis device, and the ammonia amount can be calculated. The temperature the ammonia is released at is proportional to the surface energy. Accordingly, the location of the temperature for the peak of the release is indicative of the acid strength of the surface, wherein a higher temperature indicates a higher acidity. This may be used to perform relative measurements to other catalyst samples to determine if the relative acidity is higher or lower. Once the analysis is complete, the catalyst sample and the sample chamber may be cooled back to about 50° C. in various embodiments, the sample is cooled to room temperature, for example, about 20° C., about 22° C., or about 25° C.

At block 110, a temperature programmed reduction of the catalyst with hydrogen is performed. In this step, a gas blend of about 5 vol. % in argon is flowed over the catalyst sample. While the gas is flowed over the catalyst sample, the temperature is increased from about 50° C. to about 800° C. In some embodiments, the maximum temperature may be between about 600° C. and about 900° C. Hydrogen will be consumed from the gas flow as it reduces the metal in the catalyst. The number of peaks in the temperature programmed reduction may be used to determine the number of different types of active sites.

At block 112, the catalyst sample in the cell is again treated with ammonia to react with the acidic sites. The conditions for the treatment may be the same as described with respect to block 106. After H2-TPR, the metals are reduced, and re-dispersion may occur. The re-dispersed metal may migrate to acid sites and cover some of the sites. As the temperature programmed desorption has been performed, the uncovered acidic sites are once again open for reaction. Accordingly, the conditions for the ammonia treatment may be modified. In some embodiments, the concentration of the ammonia in the gas blend with the inert gas may be increased or decreased, or the temperature of the treatment may be changed, or both.

At block 114, the temperature programmed desorption of the ammonia is repeated. The conditions of the desorption may be the same as described with respect to block 108.

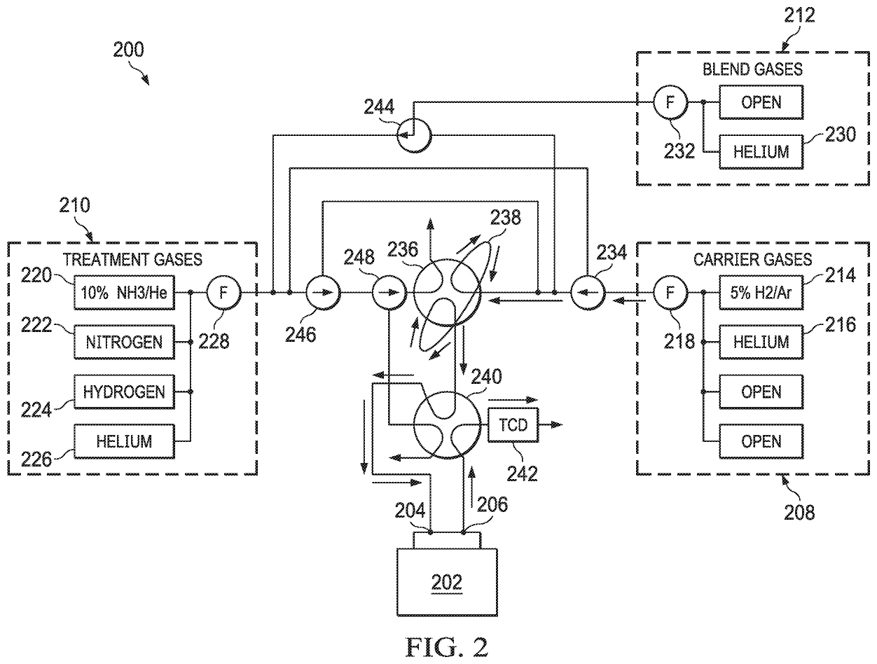

FIG. 2 is a block diagram of a chemisorption unit 200 that may be used in embodiments. The chemisorption unit 200 includes a sample chamber 202 into which the sample tube holding the catalyst sample is placed. One end of the sample tube is coupled to a gas inlet port 204 and the other end of the sample tube is coupled to a gas outlet port 206. Accordingly, gases introduced into the sample tube flow through over the catalyst sample in the sample tube. The sample chamber 202 is insulated and includes a high accuracy heating system, for example, to allow the temperature of the sample tube to be accurately ramped from about room temperature, for example, about 20° C., to about 1500° C. The temperature increase rate of the ramp may be about 5° C., about 10° C., about 20° C., or about 50° C. A slower rate may provide a more accurate measurement, but may extend the time for the test.

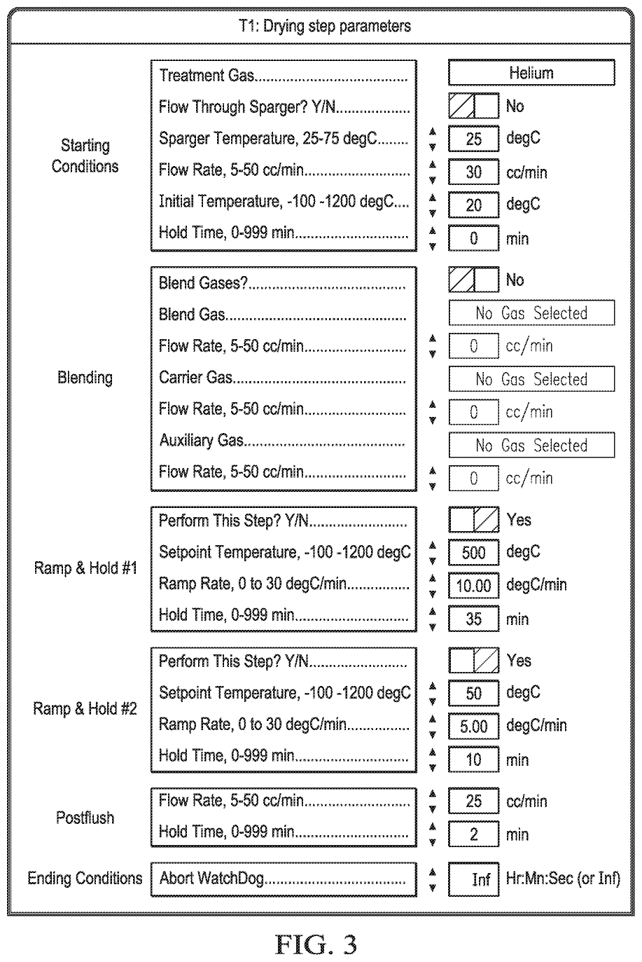

A number of gases may be flowed through the sample tube in the sample chamber, including carrier gases 208, treatment gases 210, and blend gases 212. Any number of arrangements of the gases may be used in the chemisorption unit 200. In this embodiment, and as described further with respect to FIGS. 3-8, the carrier gases (CG) 208 include a gas blend of 5% H2/Argon CG 214 and helium CG 216. The carrier gases 208 may be used during temperature programmed desorption (helium CG 216) or during the temperature programmed reduction, for example, using the gas blend of 5% H2/Argon CG 214. A CG flow controller 218 is used to control the flow of the carrier gases 208. In some embodiments, the CG flow controller 218 is used to stop the flow of carrier gas, for example, to allow treatment gases 210, blend gases 212, or both to flow through the sample to in the sample chamber 202.

The treatment gases (TG) 210 include, in this embodiment, 10% NH3/He TG 220, nitrogen TG 222, hydrogen TG 224, and helium TG 226. In some embodiments, treatment gases 210 are used for treating a sample with ammonia, for example, in a blend with a blend gas 212. In some embodiments, the treatment gases include a blended ammonia gas, such as the 10% NH3/He TG 220. In some embodiments, the hydrogen TG 224 is used in a blend for the temperature programmed reduction of a catalyst sample in the sample tube placed in the sample chamber 202. A TG flow controller 228 is used to control the flow of the treatment gases 210, for example, to stop the flow of treatment gases 210, or to proportion the flow of one of the treatment gases 210 with one of the blend gases 212 to form a treatment blend, such as 10% ammonia TG 220 with 90% helium from the blend gases 212.

The blend gases (BG) 212 include, in this embodiment, helium BG 230, which may be used to dilute and ammonia flow treating a catalyst sample prior to temperature programmed desorption. A BG flow controller 232 is used to control the flow of the blend gases 212.

A number of valves may be used to the system to allow flow and blending of the various gases used for measuring the chemisorption. These valves may be operated by solenoids under the control of the instrument to allow full automation of the test procedure. For example, a carrier gas valve 234 may be lined up to allow flow from the carrier gases 208 to a gas blending valve 236, as indicated by the arrows. The gas blending valve 236 may be lined up to direct the flow from the carrier gases 208 through a jumper line 238 between two ports on the gas blending valve 236. From the gas blending valve 236, the carrier gas flow may be directed to a test valve 240.

The test valve 240 may be lined up to direct the flow of the carrier gas to the gas inlet port 204 of the sample chamber 202. This allows the carrier gas to flow through the sample tube connected between the gas inlet port 204 and the gas outlet port 206 of the sample chamber 202. From the gas outlet port 206, the carrier gas close packed to the test valve 240, which is lined up to send the flow through a thermal conductivity detector (TCD) 242.

The TCD 242 senses changes in the thermal conductivity of the gas flowing from the outlet port 206 of the sample chamber 202, by comparing the thermal conductivity against the thermal conductivity of a flow of the carrier gas, such as helium, measured by a second sensor in the TCD 242. During temperature programmed desorption, an analyte, such as ammonia, is released into the carrier gas flowing through the catalyst sample in the sample tube in the sample chamber 202. The change in the thermal conductivity of the carrier gas due to the increase in the concentration of the analyte is measured by the TCD 242. Similarly, when other analytes, such as hydrogen, are absorbed from the carrier gas flowing through the catalyst sample, the TCD 242 measures the change in the thermal conductivity of the carrier gas due to the decrease in the concentration of the analyte. The response of the TCD 242 versus the temperature is then recorded or plotted for analysis.

Other valves are included in the chemisorption unit 200 to implement the functions. These include a blend gas valve 244 that allows the blend gases 212 to flow into lines used for mixing, for example, with the treatment gases 210. A bypass valve 246 can be lined up to allow the carrier gases 208 to be directed to the sample chamber 202 without flowing through the gas blending valve 236. A treatment gas valve 248 allows treatment gases 210 and plans of treatment gases 210 with blend gases 212 to be directly sent to the sample chamber 202, for example, without passing through the gas blending valve 236.

### EXAMPLES

The block diagram of the chemisorption unit 200 provides an example of a unit that may be used to implement the techniques described herein. An example of an implementation of the Techniques using this unit is shown in FIGS. 3-8. These figures are screenshots of a control parameter screen, showing the control parameters used for each step of the analysis.

FIG. 3 is a screenshot of the parameters used for the drying step (T1), in accordance with an embodiment. During the drying step, no treatment gases or blending gases are used, with a flow of the helium carrier gas used to sweep water, and other impurities, out of the catalyst sample. In this example, a first temperature ramp and hold is performed to a set point temperature of about 500° C. at a rate of about 10° C./min. The set point temperature is been held for about 35 min. A second temperature ramp and hold is used to cool the catalyst sample back to about 50° C., at a ramp rate of about 5° C./min.

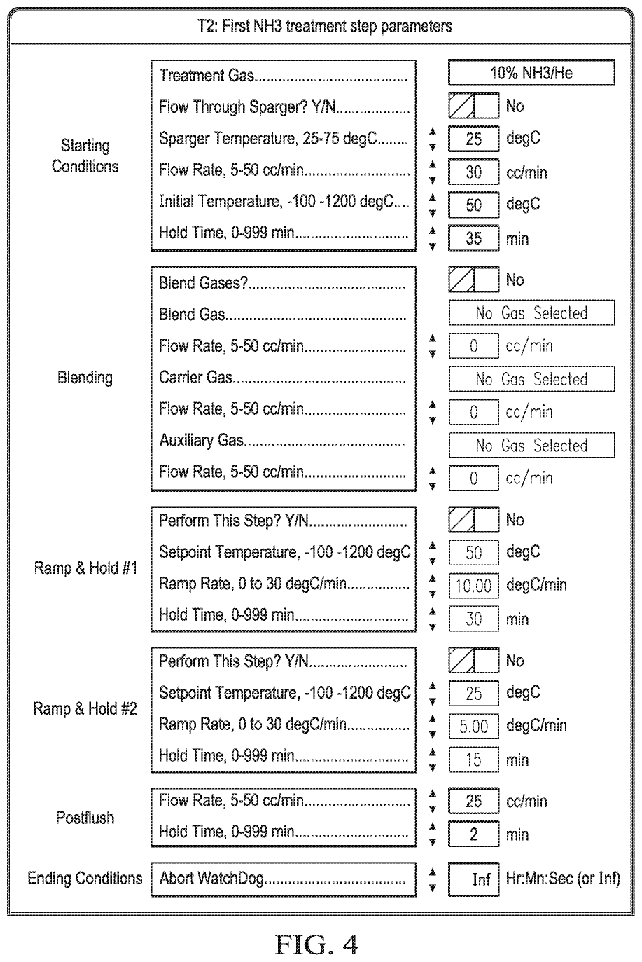

FIG. 4 is a screenshot of the parameters used for treating the catalyst sample with ammonia (T2), in accordance with an embodiment. The treatment gas is a 10% NH3/He blend, which is flowed through the sample at a rate of about 30 cc/min at about 50° C. for about 35 minutes after the treatment is completed, a postflush flow of carrier gas is passed through the catalyst sample at a flow rate of about 25 cc/min. for about two minutes.

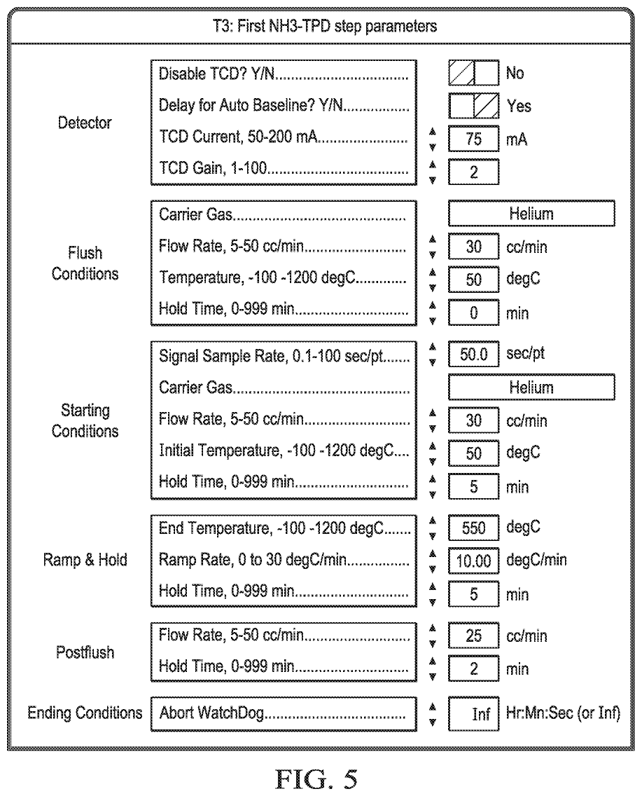

FIG. 5 is a screenshot of the parameters used for performing the first temperature programmed desorption of ammonia (T3), in accordance with an embodiment. To collect data, the thermal conductivity detector is enabled. The TCD parameters include setting an automatic baseline, running at a TCD current of about 75 mA, and a TCD gain of 2. An initial flush is performed using the helium carrier gas at a flow rate of about 30 cc/min at a temperature of about 50° C.

During the data collection, the signal from the TCD is recorded at a rate of about one data point every 50 seconds. The flow rate of the carrier gases set to about 30 cc/min. A temperature ramp is used, wherein the initial temperature is set to about 50° C. and the final temperature is set to about 550° C., with a ramp rate of about 10° C. The sample is held at the maximum temperature for about 5 minutes. A postflush flow of carrier gas is passed through the sample tube at about 25 cc/min for about two minutes.

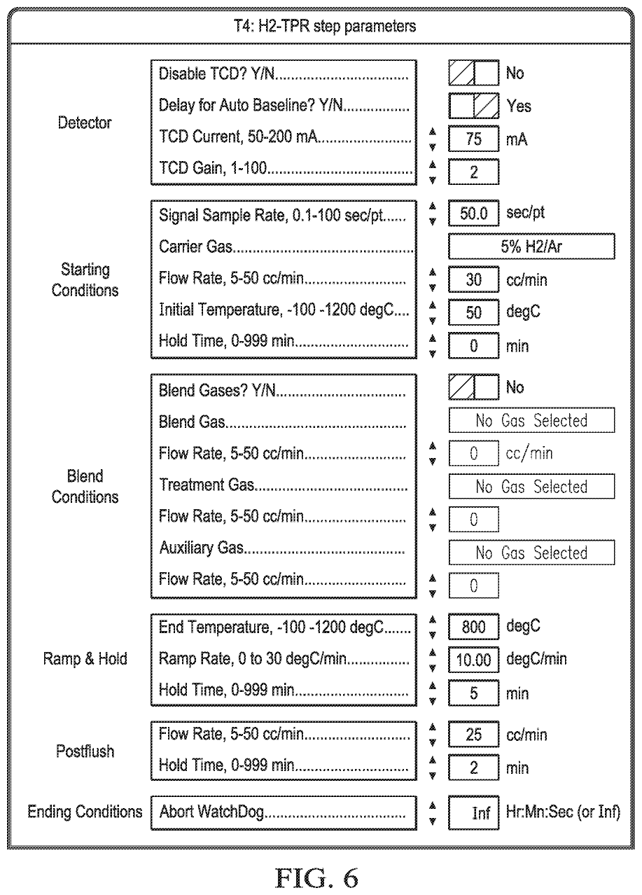

FIG. 6 is a screenshot of the parameters used for the hydrogen temperature program reduction of the catalyst sample (T4), in accordance with an embodiment. The thermal conductivity detector is enabled, using a current of about 75 mA and a gain of two. The signal sample rate is set to about 50 seconds between each data point collected. For this test, the carrier gas is blend of 5% H2/Ar. A temperature ramp is used, where the initial temperature is set to about 50° C., and the final temperature is set to about 800° C., with a ramp rate of about 10° C. A hold time of about 5 minutes at about 800° C. is used before a postflush flow of carrier gas.

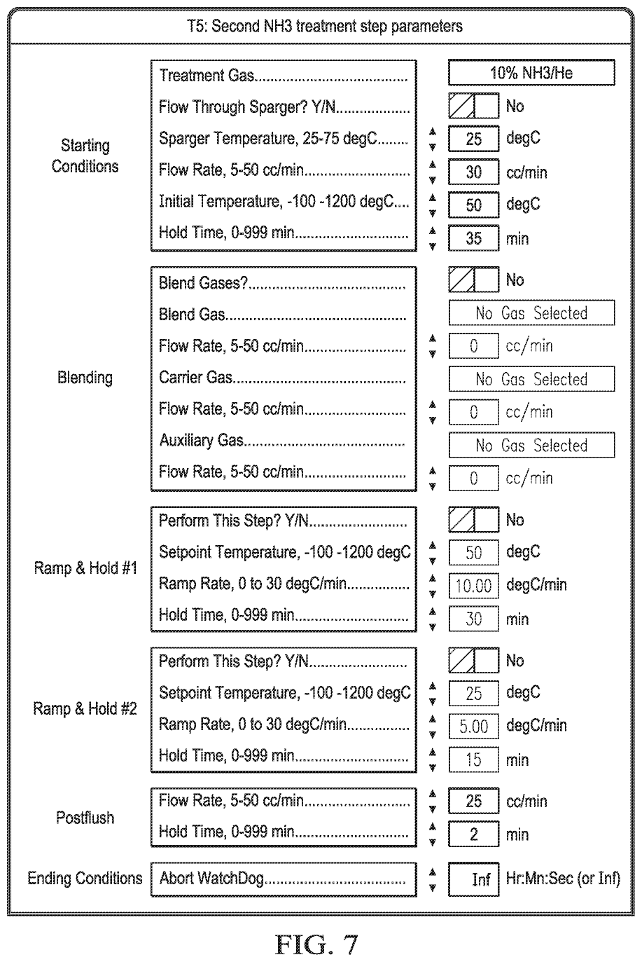

FIG. 7 is a screenshot of the parameters used for the second ammonia treatment step of the catalyst sample (T5), in accordance with an embodiment. In this example, the parameters are the same as those used for the first ammonia treatment step, described with respect to FIG. 4.

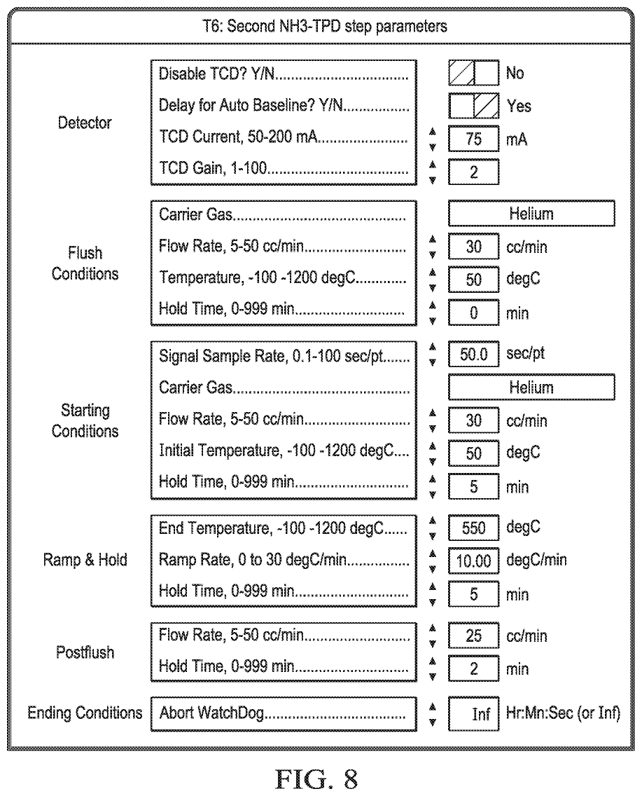

FIG. 8 is a screenshot of the parameters used for performing the second temperature programmed desorption of ammonia (T3), in accordance with an embodiment. In this example, the parameters at the same as those used for performing the first temperature programmed desorption of ammonia, described with respect to FIG. 5.

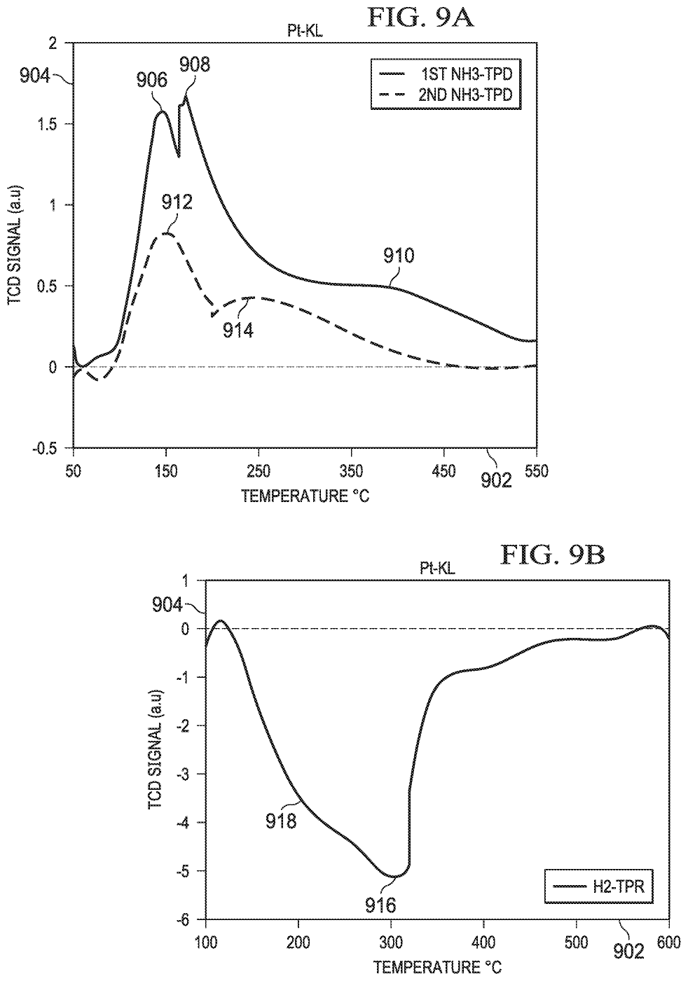

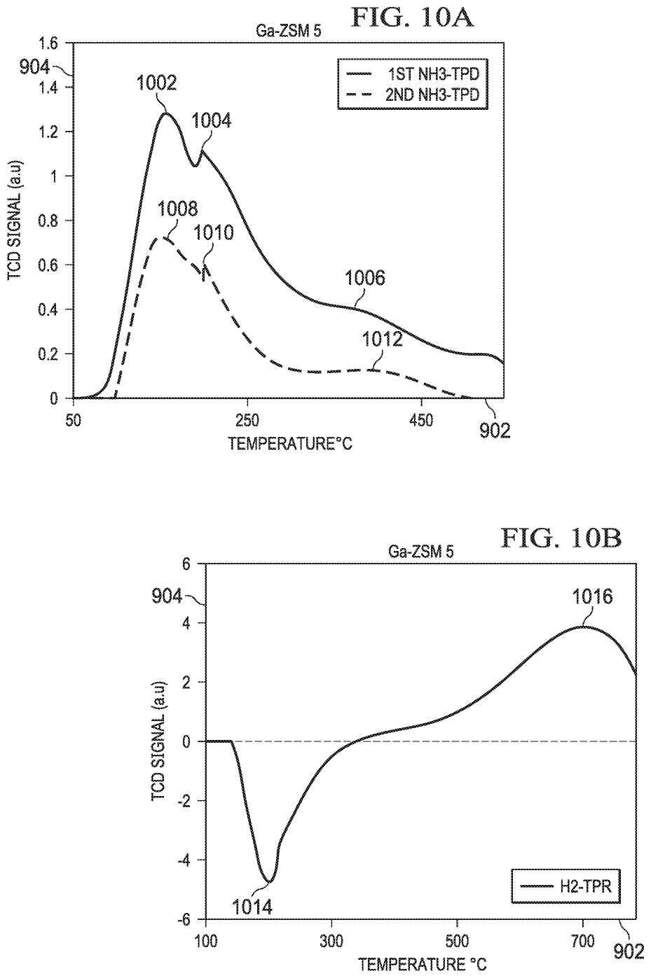

Using the parameters above, data was collected for two catalyst samples, a Pt-KL, and Ga-ZSM 5. The results for this catalyst samples are shown in FIGS. 9A-10B. In these plots the x-axis represents temperature 902 in centigrade and the y-axis represents the thermal conductivity detector (TCD) signal 904.

FIG. 9A is a plot of the first and second temperature programmed desorption curves for ammonia for the Pt-KL catalyst sample. From the signal intensity, a measurement may be made of the amount of ammonia desorbed at a particular temperature. As the ammonia is being desorbed from the catalyst sample, the peaks are positive in intensity. Ammonia is a basic gas, and thus, is absorbed by acidic sites, with the strength of the absorption dependent on the acidity of the site. Accordingly, the intensity of the TCD signal 904 at each temperature indicates the amount of ammonia released, and, therefore, the amount of acidic sites corresponding to a surface energy at that temperature. As a result, peaks may be integrated to determine the amount of acidic sites.

Peak location can be used to determine the acid strength, as the peak location corresponds to the affinity between the ammonia and the acidic sites. Peaks at lower temperatures, indicating that ammonia is released more easily, correspond to weak acidity. Peaks at higher temperatures correspond to strong acidity. Thus, from the peak location, weak acid sites and strong acid sites are differentiated. The analysis may be performed by comparing the peak locations and amounts between the first and second thermal programmed desorption of ammonia. Other comparisons may be made between the catalyst sample being tested and previous catalyst samples. Based on the specific applications for the catalyst, the strength of the acidity may be based on comparisons and temperatures. In the examples described herein, weak acidity is defined as desorption at less than about 200° C., for the location of the temperature peaks in the NH3-TPD plots).

In the first temperature program desorption of ammonia, three desorption peaks 906, 908, and 910 were measured. Integration of these peaks gave a total acidity of about 10,958 mmol/g (millimole per gram). After the temperature program reduction of hydrogen, shown in FIG. 9B. The second temperature program desorption of ammonia measured just two peaks, 912 and 914, both of which had shifted to lower temperatures, and thus, lower acidity. Integrating under the peaks of the second picture program desorption of ammonia gave a total acidity of 5518 mmol/g. This means, during metal reduction, some metals are dispersed, and cover some of acidic sites. As a result, the total acidity decreased.

FIG. 9B is a plot of the temperature programmed reduction of the Pt-KL catalyst. The peak 916 of the temperature programmed reduction was at about 300° C. However, a second peak 918, merged with the peak 916, may be present, indicating two types of catalytic sites. The peaks, 916 and 918, are negative indicating the removal of hydrogen from the carrier gas stream.

FIG. 10A is a plot of the first and second temperature program desorption of ammonia from a Ga-ZSM 5 catalyst. In the initial temperature programmed desorption, three peaks 1002, 1004, and 1006 were measured. After the temperature programmed reduction, the second temperature programmed desorption of ammonia also measured three peaks 1008, 1010, and 1012. For the sample, a smaller shift to lower acidity was seen, however, the reduction in the total number of acid sites was similar to that of the Pt-KL catalyst. Integration of the peaks indicated a decrease from a total acidity of about 16,957 mmol/g to a total acidity of about 7700 mmol/g. This also indicates a re-dispersal of the metal during the temperature programmed reduction, wherein the metal then covers some of the active sites.

FIG. 10B is a plot of the temperature programmed reduction of the Ga-ZSM 5 catalyst. In this plot, a negative peak 1014 was detected at about 200° C. and a positive peak 1016 was detected at about 700° C. This indicates that some of the hydrogen that has been chemisorbed onto the catalyst at lower temperatures is released at higher temperatures. Accordingly, the total amount of hydrogen absorbed by the catalyst is offset by the amount released from the catalyst. The results obtained from these plots is shown in table 1.

TABLE 1
NH3-TPD, and H2-TPR results of Pt/KL and Ga/ZSM-5

| | Pt/KL | Ga/ZSM-5 |
|---|---|---|
| NH3-TPD before H2 reduction | | |
| Total acidity, mmol/g | 10958 | 16957 |
| Week acidity*, mmol/g | 4164 | |
| Strong acidity**, mmol/g | 6793 | |
| NH3-TPD after H2 reduction | | |
| Total acidity, mmol/g | 5518 | 7700.5 |
| Week acidity*, mmol/g | 2593 | 4105 |
| Strong acidity**, mmol/g | 2925 | 3594 |
| H2-TPR | | |
| Consumed H2, mmol/g | 356070 | 9859 |

*NH3 desorbed at <200° C.
**NH3 desorbed at >200° C.

Other implementations are also within the scope of the following claims.

What is claimed is:

1. A method for characterizing a catalyst in a chemisorption unit, comprising:
treating a catalyst sample with a gas blend comprising ammonia in an inert gas;
performing a first temperature programmed desorption (TPD) to desorb the ammonia from the catalyst sample;
performing a temperature programmed reduction (TPR) of the catalyst sample with hydrogen;
treating the catalyst sample after the TPR with a gas blend comprising ammonia in an inert gas; and
performing a second temperature programmed desorption (TPD) to desorb the ammonia from the catalyst sample.

2. The method of claim 1, comprising:
loading the catalyst sample in the chemisorption unit; and
drying the catalyst sample by flowing the inert gas over the catalyst sample while ramping a temperature of the catalyst sample from about 50° C. to about 500° C. at about 10° C./min.

3. The method of claim 1, wherein treating the catalyst sample with a gas blend comprises flowing a blend of 10% ammonia in helium at a flow rate of about 30 cc/min for about 35 minutes at about 50° C.

4. The method of claim 1, wherein performing the first temperature programmed desorption comprises:
flowing a carrier gas over the catalyst sample, wherein the carrier gas comprises helium;
ramping a temperature of the catalyst sample from about 50° C. to about 550° C. at about 10° C./min; and
collecting data from a thermal conductivity detector to measure ammonia desorbed at a temperature during the ramping.

5. The method of claim 1, comprising measuring an amount of the ammonia desorbed from the catalyst sample during the first TPD versus time by plotting a response from a thermal conductivity detector versus temperature.

6. The method of claim 5, comprising measuring a total acidity of the catalyst sample by integrating an area of a measured response from the thermal conductivity detector.

7. The method of claim 5, comprising determining a relative acid strength of the catalyst sample by comparing peak locations of the ammonia released from other catalyst samples.

8. The method of claim 5, comprising cooling the catalyst sample under a flow of the inert gas.

9. The method of claim 1, comprising measuring an amount of hydrogen adsorbed by the catalyst sample during the TPR by measuring an amount of hydrogen removed from a carrier gas.

10. The method of claim 1, wherein performing the TPR of the catalyst sample with hydrogen comprises:
flowing a carrier gas over the catalyst sample, wherein the carrier gas comprises a gas blend of 5% hydrogen in argon;
ramping the temperature of the catalyst sample from about 50° C. to about 800° C. at about 10° C./min; and
collecting data from a thermal conductivity detector to measure an amount of hydrogen removed from the carrier gas at a temperature during the ramping.

11. The method of claim 10, comprising determining an activity of the catalyst sample by integrating peaks of the hydrogen adsorbed during the TPR.

12. The method of claim 10, comprising determining a number of active sites of the catalyst sample by determining a number of peaks in a plot of the TPR.

13. The method of claim 10, comprising dispersing at least a portion of metal sites by performing the TPR.

14. The method of claim 1, wherein treating the catalyst sample after the TPR comprises flowing a gas blend comprising 10% ammonia in helium over the catalyst sample at a flow rate of about 30 cc/min for about 35 minutes at about 50° C.

15. The method of claim 14, wherein performing the second temperature programmed desorption comprises:
flowing a carrier gas over the catalyst sample, wherein the carrier gas comprises helium;
ramping the temperature of the catalyst sample from about 50° C. to about 550° C. at about 10° C./min; and
collecting data from a thermal conductivity detector to measure ammonia desorbed at a temperature during the ramping.

16. The method of claim 14, comprising measuring an amount of the ammonia desorbed from the catalyst sample during the second TPD versus time by plotting a response from a thermal conductivity detector versus temperature.

17. The method of claim 16, comprising measuring a total acidity of the catalyst sample by integrating an area of a measured response from the thermal conductivity detector.

18. The method of claim 16, comprising determining a relative acid strength of the catalyst sample by comparing peak locations of the ammonia released from other catalyst samples.

19. The method of claim 1, comprising determining a change in acid strength of the catalyst sample by comparing results from the second temperature programmed desorption to results of the first temperature programmed desorption.
